(19) United States
(12) Patent Application Publication
(10) Pub. No.: US 2025/0090859 A1
(43) Pub. Date: Mar. 20, 2025

(54) SYSTEM AND METHOD FOR LOCALIZED THERAPEUTIC TREATMENT

(71) Applicants: Suehyun CHO, Los Angeles, CA (US); Florent CROS, Los Angeles, CA (US); Alex KISELYOV, San Diego, CA (US); Michael SHPIGELMACHER, Los Angeles, CA (US); BIONAUT LABS LTD., Herzliya (IL)

(72) Inventors: Suehyun CHO, Los Angeles, CA (US); Florent CROS, Los Angeles, CA (US); Alex KISELYOV, San Diego, CA (US); Michael SHPIGELMACHER, Los Angeles, CA (US)

(73) Assignee: BIONAUT LABS LTD., Herzliya (IL)

(21) Appl. No.: 18/562,260

(22) PCT Filed: May 20, 2022
(86) PCT No.: PCT/US2022/030221

§ 371 Date: Nov. 17, 2023

Related U.S. Application Data
(60) Provisional application No. 63/191,497, filed on May 21, 2021.
(60) Provisional application No. 63/191,515, filed on May 21, 2021.
(60) Provisional application No. 63/191,418, filed on May 21, 2021.
(60) Provisional application No. 63/191,454, filed on May 21, 2021.
(60) Provisional application No. 63/213,120, filed on Jun. 21, 2021.

(51) Int. Cl. A61N 5/06

(52) U.S. Cl. CPC A61N 5/062; A61N 5/0601; A61N 2005/065

(57) ABSTRACT

Provided are systems and miniature devices configured to navigate within a patient to a location therewithin for delivering to induce a localized therapeutic effect, such as the delivery of catalyzing energy and/or for the conversion of a prodrug to a pharmaceutically active drug. Further provided are various methods of treatment using such systems and devices.

### FIELD OF THE INVENTION

The presently disclosed subject matter relates to systems and miniature devices configured to navigate within a patient to a location therewithin to induce a localized therapeutic effect, such as the delivery of catalyzing energy and/or for the conversion of a prodrug to a pharmaceutically active drug.

### BACKGROUND OF THE INVENTION

Therapeutics and diagnostics have traditionally been administered to patients via various routes, including orally, nasally, intravenously, subcutaneously, intramuscularly, using syringes, pills, salves, sprays, solutions, and so on. These traditional routes and means for accessing a patient's body suffer various several major drawbacks.

First, global administration of a therapeutic is not always desirable. Often, due to risk of adverse side effects, it would be preferable to deliver a therapeutic only to a desired target, e.g., a tumor. Further, some therapeutics are very expensive, and it would be more efficient use of a valuable resource to target the expensive therapeutic only to where it is needed in the patient's body.

Remote control of medical devices moving inside the human body can be useful for a variety of purposes, including delivery of therapeutic payloads, diagnostics, or surgical procedures. In many medical applications, it would be useful to use a mobile medical device to move within a living organism. For example, it may be desirable to move an internal device through tissue to a particular desired anatomic location to activate a drug. Such devices may include microscale or nanoscale robots, medical tools, “smart pills,” etc.

Such devices may be able to move in the body either through self-propulsion or an external propulsion mechanism. Accurate location and tracking of such devices may be necessary to ensure their proper functioning at the right anatomical location, and more specifically accurate delivery of the therapeutic payloads and/or diagnostics substances.

### SUMMARY OF THE INVENTION

According to an aspect of the presently disclosed subject matter, there is provided a system configured to facilitate treatment at a target site in a patient, the system comprising:
at least one miniature device configured to be maneuvered to the target site under manipulation by an external non-contact force, the miniature device comprising an externally triggered energy supply; and
a driving apparatus configured for creating the external non-contact force to manipulate the miniature device to move within the patient.

In some embodiments, the system further comprises a triggering apparatus configured to remotely trigger the energy supply to produce the catalyzing dose of energy, wherein the energy supply is configured to produce a catalyzing dose of energy to induce a therapeutic effect at the target site, thereby facilitating treatment.

In some embodiments, the miniature device carries a prodrug activating agent and is configured to facilitate conversion of the prodrug into the therapeutic agent, thereby facilitating treatment. Accordingly, the activating agent may serve as a catalyst to facilitate treatment.

In some embodiments, the system comprises a triggering apparatus configured to remotely trigger the energy supply to produce the catalyzing dose of energy; and the miniature device also carries a prodrug activating agent which facilitates conversion of the prodrug into the therapeutic agent, thereby facilitating the treatment.

According to another aspect of the presently disclosed subject matter, there is provided a system configured to facilitate treatment by a therapeutic agent at a target site in a patient, said therapeutic agent being formed by conversion of a prodrug, the system comprising:
at least one miniature device configured to be maneuvered to the target site under manipulation by an external non-contact force, the miniature device carrying an activating agent that converts the prodrug into the therapeutic agent;
a driving apparatus configured for creating the external non-contact force to manipulate the miniature device to move within the patient.

In some embodiments, the driving apparatus is further configured to manipulate the miniature device to selectively release one or more guide substances and/or recognition substances along a path within the patient, wherein the recognition substance has a high affinity for the guide substance. In some such embodiments, the systems further comprise one or more delivery units, each comprising the therapeutic agent and recognition substance. In some embodiments, the miniature device is configured to release the guide substance according to a predetermined program. In some embodiments, the miniature device is configured to selectively vary the density of the guide substance released along the path. For example, the miniature device is configured to increase the density of the guide substance released as it approaches the target site.

In such embodiments wherein the driving apparatus is configured to manipulate the miniature device to selectively release one or more guide substances and/or recognition substances, one of the guide and recognition substances comprises streptavidin, with the other of the guide and recognition substances comprising biotin. One of the guide and recognition substances may comprise chemokine ligand 2 (CCL2), with the other of the guide and recognition substances comprising chemokine receptor type 2 (CCR2). It will be appreciated that the guide and/or recognition substance may comprise a chemical in the sense that it is configured to express it. In some embodiments, the recognition substance is connected to the therapeutic agent via a cleavable linker. The cleavable linker may be a labile chemical bond susceptible to cleavage via an endogenous stimulus. The endogenous stimulus may be selected from an acidic environment, a reduction-oxidation reaction, and an enzyme. The cleavable linker may be a labile chemical bond susceptible to cleavage via an external stimulus. The external stimulus may be selected from an ultrasound signal, an optical signal, and an electrical signal. The recognition substance may be connected to the therapeutic agent via a non-cleavable linker. The therapeutic agent may constitute or comprise the recognition substance. Each delivery unit may be configured to release the therapeutic agent in response to one or more exogenous or endogenous stimuli, for example according to examples in which the recognition substance comprises a cell. The therapeutic agent may comprise at least one selected from small molecules, peptides, peptoids, oligonucleotide sequences, nucleic acids, oncolytic viruses, endogenous cells, and/or engineered cells. The recognition substance may be selected from a molecule and a cell.

The system may be configured to facilitate treatment by a therapeutic agent configured to produce a therapeutic effect in the presence of the catalyzing dose of energy. For example, the therapeutic agent is configured to be photoactivated; is a photosensitizing agent; and/or has a photocleavable moiety. Alternatively, the therapeutic agent comprises one or more molecules that assume an active conformation, assembly, aggregation, and/or modification upon exposure to light. The system may comprise the therapeutic agent.

The catalyzing dose of energy may be configured to trigger a physiological process in the patient, wherein the physiological process facilitates the therapeutic effect. For example, the process may be selected from enhanced local pharmacokinetics, absorption, rupture of a physiological barrier, distribution, permeability, proliferation, differentiation, adhesion, motility, or a combination thereof.

In some embodiments, the catalyzing dose of energy is light energy. In some embodiments, the triggering apparatus is configured to direct the energy supply to vary the energy level produced.

In some embodiments, the miniature device further comprises a drive portion affixed to the energy supply and configured to interact with the external non-contact force to effect maneuvering, and the drive portion is configured to separate from the energy supply. The triggering or driving apparatus may be configured to direct the separation of the drive portion and energy supply.

In some embodiments, the system may further comprise the prodrug.

The prodrug activating agent may facilitate conversion of the prodrug into the therapeutic agent by directly interacting with the prodrug. The activating agent may comprise an enzyme and/or an enzymatically active oligonucleotide.

The activating agent may encode an auxiliary activating agent, where the auxiliary activating agent is configured to directly interact with the prodrug to facilitate its conversion into the therapeutic agent. An extracellular, intracellular, and/or intranuclear process may express the auxiliary activating agent encoded by the activating agent. The activating agent may comprise an enzyme precursor, an oligonucleotide precursor, and/or a protease precursor.

The system may also comprise a vector configured for cellular delivery of the activating agent.

The vector may be selected from an adeno-associated virus (AAV), a human immunodeficiency virus, a human papillomavirus sequence, one or more small molecules, one or more lipids, a peptide sequence, and a recognition sequence.

The activating agent may be selected from an endogenous or a non-endogenous human enzyme, a pro-enzyme, a construct encoding an active enzyme, and a cell/nuclear delivery sequence (e.g., a small molecule, a lipid, a specific receptor affinity sequence, an AAV-based delivery vector).

The activating agent may be, and/or may encode, a kinase, a phosphatase, a peptidase, a ligase, a lyase, a hydrolase, a protease, a deacetylase, a phosphodiesterase, an esterase, an amidase, a reductase, a phospholipase, or a cytochrome.

The activating agent may be carried on an exterior surface of the miniature device.

The miniature device may comprise a coating configured to dissipate at the target site at least partially under one or more predetermined conditions, thereby releasing the activating agent. At least one of the predetermined conditions may be selected from a magnetic, ultrasound, radiofrequency (RF), optical, electric, and a combination of one or more thereof, the system further comprising a disruption apparatus configured to establish the predetermined condition at the target site.

At least one of the predetermined conditions may be selected from dissolution, dispersion, decomposition, metabolism, a pH change, a redox reaction, and the presence of one or more enzymes.

In some embodiments, the coating may surround the activating agent.

In some embodiments, the activating agent may be mixed with the material of the coating.

The miniature device may be configured to controllably release the activating agent.

According to other aspects of the presently disclosed subject matter, there is provided a system to facilitate treatment at a target site in a patient, comprising a miniature device configured to be maneuverable to a target site, e.g., under manipulation by an external non-contact force, the miniature device comprising at least one element configured to induce a therapeutic effect at the target site.

The element may be configured to induce a therapeutic effect by a substance (e.g., a therapeutic or other agent) administered to the patient, and which is inactive when not so induced.

The element may comprise an activating agent configured to facilitate conversion of a prodrug into a therapeutic agent at the target site, as described herein.

The element may comprise an energy source configured to produce a catalyzing dose of energy to induce the therapeutic effect at the target site, e.g., by activating a therapeutic agent and/or by triggering a physiological process in the patient at the target site.

According to other aspects of the presently disclosed subject matter, there is provided a method for providing localized treatment at a target site in a patient using the above system.

The drive portion may be affixed to the energy supply by an adhesive material. The adhesive material may be configured to be disrupted under a predetermined condition, thereby separating the carrier portion from the drive portion. The predetermined condition may be selected from melting, dissolving in a solvent, chemically induced matrix rupture, exposure to radio and/or ultrasound waves, and exposure to near infrared frequency.

The miniature device may comprise the therapeutic agent and disrupting the adhesive material releases the therapeutic agent. The adhesive material may be mixed with the therapeutic agent.

The adhesive material may be insulated from the environment by a bioerodible material configured to delay the disruption of the adhesive material.

The miniature device, e.g., the energy supply thereof, may comprise one or more anchors configured to anchor the energy supply adjacent the target site.

The non-contact force may be selected from a group including magnetic, electromagnetic, ultrasound, radio-frequency, optical, and a combination of one or more thereof.

According to another aspect of the presently disclosed subject matter, there is provided a method for providing localized treatment at a target site in a patient, the method comprising:
providing a system as described herein;
introducing the miniature device of the system into the patient at an injection site;
operating the driving apparatus of the system to navigate the miniature device to the target site; and
administering a prodrug to the patient, wherein the prodrug is converted into a therapeutic agent by an activating agent carried by the miniature device and/or by a catalyzing dose of energy from the miniature device, thereby effecting treatment at the target site.

According to another aspect of the presently disclosed subject matter, there is provided a method for providing localized treatment at a target site in a patient, the method comprising:
providing a system as above;
introducing a miniature device of the system into the patient at an injection site;
operating the driving apparatus of the system to navigate the miniature device to the target site; and
operating the triggering apparatus to trigger the miniature device to produce a catalyzing dose of energy to induce a therapeutic effect at the target site.

The method may further comprise administering to the patient a therapeutic agent that is configured to produce a therapeutic effect in the presence of the catalyzing dose of energy.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1B schematically illustrate embodiments of systems described herein. FIG. 1A illustrates an embodiment wherein the system delivers catalyzing energy. FIG. 1B illustrates an embodiment wherein the system delivers a prodrug activating agent.

FIG. 2 schematically shows a triggering circuit of a miniature device of the system in FIG. 1A.

FIG. 3 schematically illustrates a harvest circuit of a miniature device of the system in FIG. 1A.

FIG. 4 illustrates an example of a miniature device of the system illustrated in FIG. 1A.

FIGS. 5A-5C are examples of miniature devices of the system illustrated in FIG. 1B. FIGS. 5A-5B depict embodiments with coatings. FIG. 5C depicts an embodiment having an internal chamber containing a payload within.

FIGS. 6A-6B are block diagrams illustrating methods of localized treatment of a patient at a target site in a patient using the system illustrated in FIG. 1. FIG. 6A illustrates a method of localized treatment using a system such as illustrated in FIG. 1A. FIG. 6B illustrates a method of localized treatment using a system such as illustrated in FIG. 1B.

FIG. 7 illustrates a modification of the system illustrated in FIGS. 1A-1B.

FIGS. 8A-8D illustrate photographic bioluminescence data from mice treated as described in the Examples below. FIG. 8A illustrates the negative control. FIGS. 8B, 8C, and 8D illustrate three experimental mouse specimens dosed in the right brain hemisphere.

FIGS. 9A-9D illustrate photographic bioluminescence data from mice treated as described in the Examples below. FIGS. 9A and 9C depict the same individual negative control mouse specimen, while FIGS. 9B and 9D depict the same individual experimental mouse specimen at 11 days of treatment. FIG. 9B illustrates bioluminescence of a first target in the mouse's left brain hemisphere;

FIG. 9D illustrates bioluminescence of a second target in the mouse's right brain hemisphere.

FIG. 10A-10C illustrate photographic bioluminescence data from the same mouse specimen as FIGS. 9B. 9D, at 60 days of treatment. FIG. 10A illustrates bioluminescence of a first target in the left hemisphere; FIG. 10B illustrates bioluminescence of a second target in the right hemisphere; FIG. 10C illustrates a layered composite image reflecting the bioluminescence data of FIGS. 10A and 10B.

FIG. 11 illustrates a 3-dimensional spatial representation of two different bioluminescence outputs from an experimental mouse treated using an embodiment of the system of FIG. 1B.

FIG. 12 illustrates an embodiment of a system having a guide substance.

FIG. 13 illustrates an embodiment of the miniature device of the presently described system, the miniature device having an internal chamber in which it may carry a payload.

FIG. 14 depicts a block diagram of a method of using the system with a guide substance.

FIG. 15 illustrates an exemplary route for the miniature device, wherein the miniature device delivers one or more therapeutic agent(s) to a target site in a patient's brain.

### DETAILED DESCRIPTION OF THE INVENTION

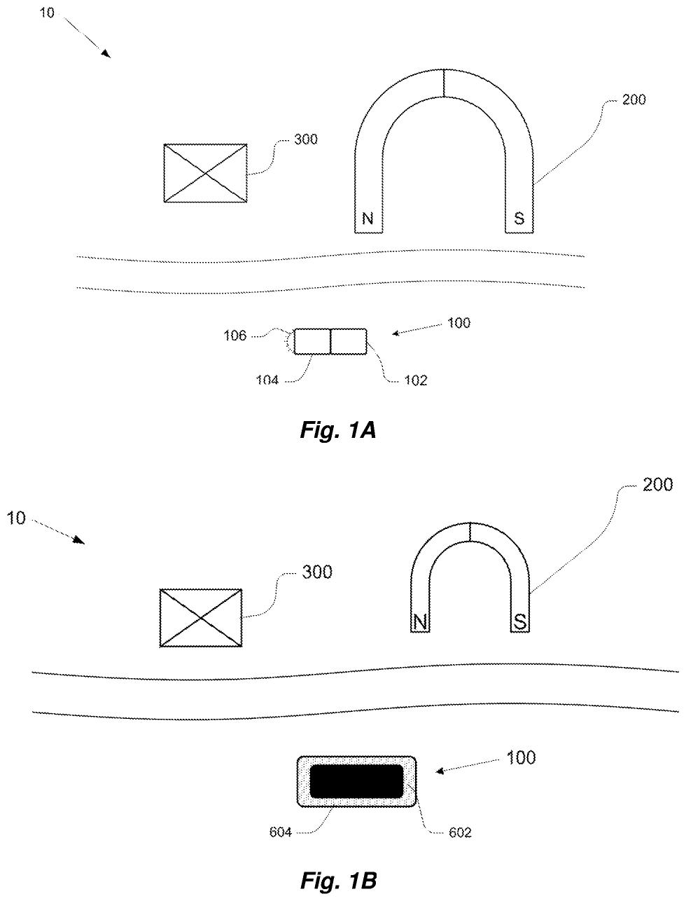

As illustrated in FIGS. 1A-1B, there is provided a system, which is generally indicated at 10, for treatment of a patient by, e.g., a therapeutic agent at (e.g., in the vicinity of; the range of what constitutes the “vicinity” may be determined by the user) a target site in a patient. The therapeutic agent may comprise, e.g., one or more chemical compounds of medicinal, diagnostic, evaluative, and/or therapeutic relevance, and in particular such therapeutic agent(s) may be characterized by being activated by exposure to an external predetermined dose of energy; and/or may be a prodrug that may be activated by an activating agent.

It will be appreciated that the term “dose” as used herein expresses that the energy conforms to one or more predetermined parameters, for example including, but not limited to, the form of energy, amplitude, duration, direction, regimen (e.g., continuous, pulsating, periodic, etc.), etc. It will be further appreciated that the therapeutic agent is inactive, or significantly less active, prior to and/or in the absence of exposure to the predetermined dose of energy.

One or more components of the system may be provided, mutatis mutandis, as described in any one or more of WO 2019/213368, WO 2019/213362, WO 2019/213389, WO 2020/014420, WO 2020/092781, WO 2020/092750, WO 2018/204687, WO 2018/222339, WO 2018/222340, WO 2019/212594, WO 2019/213368, WO 2019/005293, WO 2020/096855, WO 2020/252033, WO 2021/021800, WO 2021/092076, and PCT/US2020/65207, and U.S. Provisional application Nos. 63/012,358, 63/120,529, 63/191,454, 63/191,418, 63/191,515, and 63/191,497, the full contents of which are incorporated herein by reference.

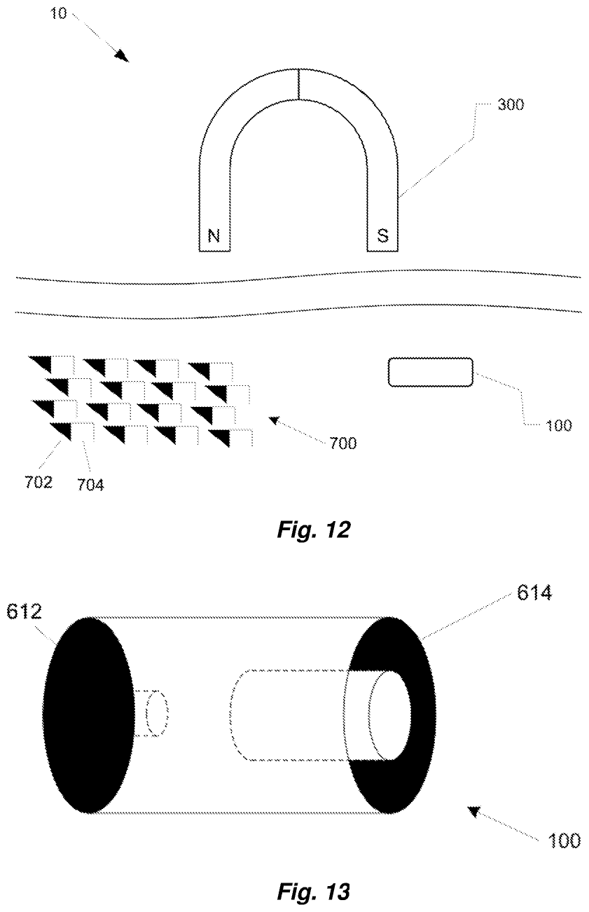

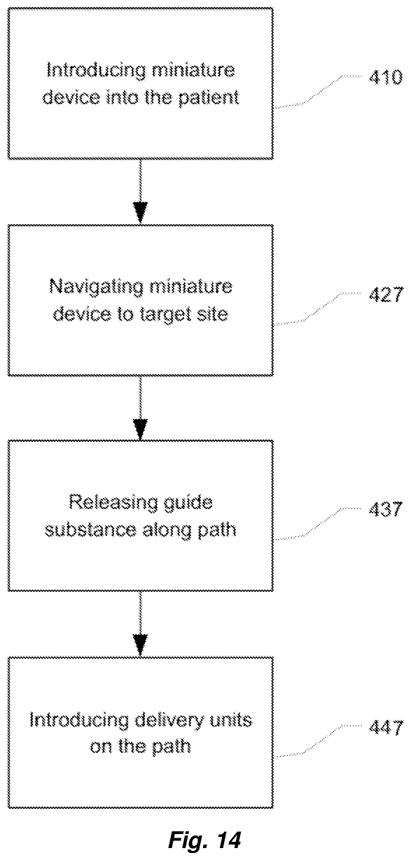

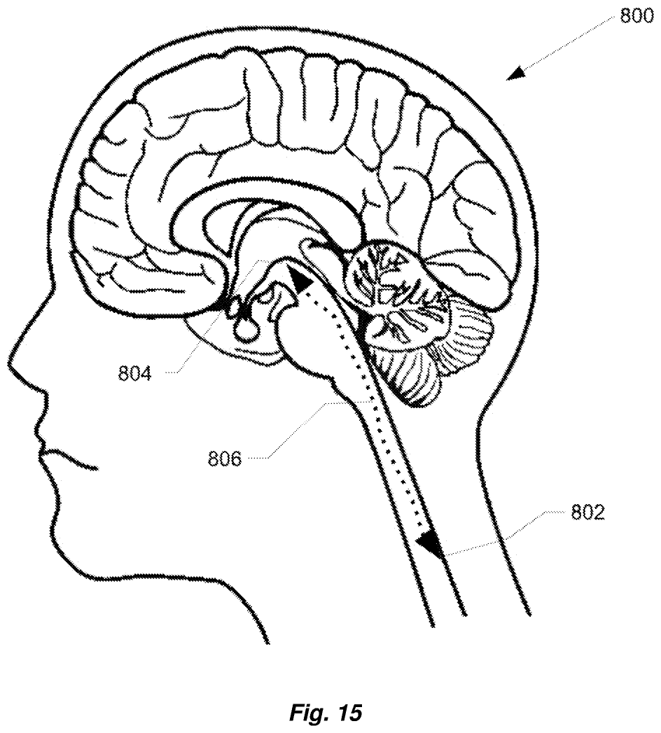

System 10 comprises miniature device 100, a driving apparatus 200, and a triggering apparatus 300. According to some examples, driving apparatus 200 and triggering apparatus 300 are embodied by a single device; however, for the sake of disclosure they will be treated herein as two separate devices. Similarly, driving apparatus 200 and triggering apparatus 300 are schematically illustrated with two different symbols in FIGS. 1A-1B and FIG. 12 for the sake of disclosure; however, as will be discussed below, they may be implemented with the same technology.

Driving apparatus 200 is configured to creating a non-contact force to manipulate the miniature device to move (i.e., to provide a motive force thereto, as well as to steer it) within a patient, for example by generating a varying magnetic field and thereby remotely, i.e., from a location exterior to a patient's body, controlling the motion of miniature device 100 within the body.

According to some embodiments, characteristics of the magnetic field, for example including, but not limited to, distance, directionality, intensity, gradient, time dependence/independence, etc., may be controlled by a user in order to remotely control the motion of miniature device 100.

It will be appreciated that while the description below refers to a miniature device controlled by a magnetic inducting apparatus, this is by way of example only and is not to be construed as limiting; the disclosed subject matter here also applies to a system in which a miniature device is remotely controlled or maneuvered by an apparatus external to the patient, in particular wirelessly.

It will be further appreciated that while herein the specification and appended claims reference is made to a therapeutic agent, in practice system 10 may be configured to deliver more than one type of therapeutic agent; the term “therapeutic agent” will be employed herein in the singular for simplicity of disclosure only, and is not to be construed as limiting any of the examples and/or embodiments disclosed or recited herein to a single therapeutic agent, mutatis mutandis.

According to some embodiments, such as shown in FIG. 1A, miniature device 100 comprises drive portion 102 composed partially or entirely of a magnetic material, and energy supply 104 connected thereto. Drive portion 102 is configured to interact with the magnetic field generated by magnetic inducing apparatus 200, thereby facilitating control of the miniature device by selectively altering the magnetic field. Energy supply 104 is configured to produce a catalyzing dose of energy to induce a therapeutic effect at the target site, thereby facilitating the treatment. The energy may be stored by energy supply 104, for example in a different form, until the dose is produced, and/or it may be configured to convert externally supplied energy, for example in a different form, into the dose.

According to examples in which the therapeutic agent is activated by exposure to an external predetermined dose of energy, energy supply 104 may be configured to produce such an energy dose.

According to some examples, the energy supply comprises a light source 106, e.g., a light-emitting diode (LED). Accordingly, the therapeutic agent may be photoactivated. According to other examples, it may be a photosensitizing agent, comprise a photocleavable moiety, and/or comprise one or more molecules that assume an active conformation, assembly, aggregation, and/or modification upon exposure to light.

Triggering apparatus 300 may be configured to remotely trigger energy supply 104 to produce the catalyzing dose of energy. This may be accomplished by any suitable means. According to some examples, triggering apparatus 300 is configured to produce a wireless signal based on a non-contact force which is of a different type than created by driving apparatus 200, e.g., if the driving apparatus creates a magnetic force to manipulate miniature device 100, the triggering apparatus may operate to trigger energy supply 104 by producing a radio-frequency signal, in order to prevent the energy supply from being triggered by a signal intended to manipulate the miniature device to move, and vice versa.

In some embodiments, such as shown in FIG. 1B, the miniature device may be configured to effectuate delivery of a prodrug activating agent. In such embodiments, miniature device 100 comprises drive portion 602 composed partially or entirely of a magnetic material and carries payload 604 comprising a prodrug activating agent, i.e., a molecule, chemical, or other suitable substance configured to facilitate conversion of the prodrug into the therapeutic agent. The activating agent may facilitate the conversion directly, i.e., by interacting with the prodrug, or indirectly, e.g., by facilitating production of an agent which directly interacts with the prodrug to convert it into the therapeutic agent. It will be appreciated that while payload 604 is illustrated in FIG. 1B as being attached to the exterior of miniature device 100, this is by way of illustration only, and is not meant to be limiting. Miniature device 100 may carry the activating agent in any suitable fashion.

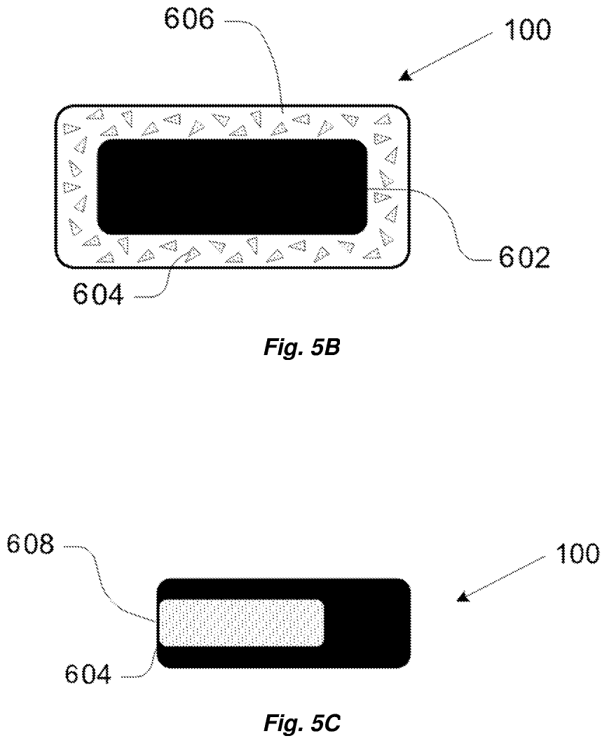

According to some examples, the activating agent is attached to the miniature device, for example covalently or non-covalently. Optionally, a coating 606 may be provided surrounding at least a portion of miniature device 100. According to some examples, such as shown in FIG. 5A, coating 606 may surround payload 604. According to other examples, such as shown in FIG. 5B, payload 604 may be mixed with the material of coating 606, e.g., which may be applied directly onto miniature device 100. Coating 606 may be configured to at least partially dissipate, for example under one or more predetermined conditions, such as, a particular temperature, pH, salinity, etc. Accordingly, the release of the activating agent, and thus the conversion of the prodrug into the therapeutic agent, may be selectively controlled. Other examples of the predetermined condition may include, but are not limited to, a magnetic condition (e.g., the presence of a magnetic signal), an ultrasound condition (e.g., the presence of an ultrasound signal), a radiofrequency condition (e.g., the presence of an RF signal), an optical condition (e.g., the presence of an optical signal), an electric condition (e.g., the presence of an electric signal), and a combination thereof. Triggering apparatus 300 may be configured to establish the predetermined condition at the target site. Alternatively, the predetermined condition may be one or more endogenous (i.e., environmental) factors, e.g., present at the target site, including, but not limited to, dissolving, dispersion, decomposition, metabolism, a change in pH (e.g., the pH at the target site is above the isoelectric point of the material of the coating), a redox reaction, and the presence or absence of one or more enzymes at the target site.

According to other examples, for example as shown in FIG. 5C, miniature device 100 may comprise an internal chamber 608 containing payload 604 therewithin. Internal chamber 608 may be opened, e.g., selectively, according to any suitable method, for example in response to an externally applied signal, in response to one or more environmental factors, etc.

The activating agent may directly facilitates conversion of a prodrug into the therapeutic agent, e.g., it may comprise an enzyme or an enzymatically active oligonucleotide. Alternatively, the activating agent may indirectly facilitates conversion of a prodrug into the therapeutic agent.

For example, the activating agent may encode an auxiliary activating agent which itself is configured to directly interact with the prodrug to facilitate its conversion into the therapeutic agent, or which itself indirectly facilitates conversion of the prodrug. The activating agent may prompt an intracellular and/or intranuclear process to express the auxiliary activating agent encoded by the activating agent. Examples of activating agents which indirectly facilitate conversion of the prodrug into the therapeutic agent include, but are not limited to, an enzyme precursor, an oligonucleotide precursor, and a protease precursor.

Payload 604 may comprise an activating agent configured to indirectly facilitate conversion of the prodrug into the therapeutic agent, one or more vectors configured for cellular delivery of the activating agent. Examples of such vectors include, but are not limited to, an adeno-associated virus, a human immunodeficiency virus, a human papillomavirus sequence, one or more small molecules, one or more lipids, a peptide sequence, and a recognition sequence.

Examples of the activating agent and/or auxiliary activating agent include, but are not limited to, a kinase, a phosphatase, a peptidase, a ligase, a lyase, a hydrolase, a protease, a deacetylase, a phosphodiesterase, an esterase, an amidase, a reductase, a phospholipase, and a cytochrome.

Triggering apparatus 300 may be configured to remotely facilitate release of the payload by causing coating 606 to dissipate, thereby releasing payload 604 comprising the activating agent. This may be accomplished by any suitable means. According to some examples, disruption apparatus 300 is configured to produce a wireless signal based on a non-contact force which is of a different type than created by driving apparatus 200, e.g., if the driving apparatus creates a magnetic force to manipulate miniature device 100, the disruption apparatus may operate to cause dissipation of coating 606 by producing a radio-frequency signal, in order to prevent a situation in which the dissipation is caused by a signal intended to manipulate the miniature device to move, and vice versa.

According to some examples, triggering apparatus 300 is further configured to vary the level (e.g., intensity) of non-contact force produced. Accordingly, a single miniature device 100 may be used to selectively release payload 604 of the activating agent at a predetermined rate, e.g., to facilitate different treatments at a target site, vary the intensity of the therapeutic effect, etc.

Triggering apparatus 300 may be configured to remotely facilitate release of the payload by causing coating 606 to dissipate, thereby releasing payload 604 comprising the activating agent. This may be accomplished by any suitable means. According to some examples, triggering apparatus 300 is configured to produce a wireless signal based on a non-contact force which is of a different type than created by driving apparatus 200, e.g., if the driving apparatus creates a magnetic force to manipulate miniature device 100, the disruption apparatus may operate to cause dissipation of coating 606 by producing a radio-frequency signal, in order to prevent a situation in which the dissipation is caused by a signal intended to manipulate the miniature device to move, and vice versa.

According to other examples, driving apparatus 200 and triggering apparatus 300 are configured to operate using the same type of non-contact force. Accordingly, miniature device 100 may be configured to differentiate between different types of signals, e.g., based on frequency, encoded signals, etc., to prevent energy supply 104 from being triggered by a signal intended to manipulate the miniature device to move, and vice versa.

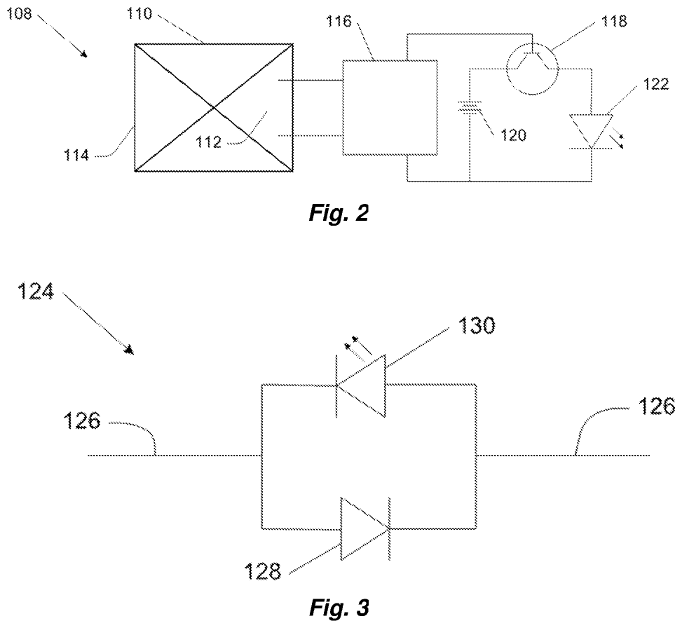

As shown in FIG. 2, in embodiments such as shown in FIG. 1A, energy supply 104 may comprise a triggering circuit 108, configured to facilitate triggering the energy supply to produce an energy dose. According to some examples, triggering circuit 108 may comprise tank circuit 110 comprising capacitor 112, inductor 114, rectifier 116, transistor 118, energy source 120, and LED 122. As is well-known in the art, the resonant frequency of tank circuit 110 is given by

$$\frac{1}{2 π \sqrt{L C}} ,$$

in which L is the inductance of inductor 114 in Henries, C is the capacitance of capacitor 112 in Farads, and the frequency is expressed in Hertz.

In operation, a signal, such as an RF signal, is produced by triggering apparatus 300 at the resonant frequency of tank circuit 110. This produces a current in tank circuit 110, which is rectified by rectifier 116, turning on transistor 118. In its “on” state, the transistor allows energy from energy source 120 to power LED 122, which produces the required dose of energy.

According to some examples, energy supply 104 is configured to harvest energy, e.g., supplied by triggering apparatus 300, to produce the required energy dose, for example being a different form of energy as that supplied. As shown in FIG. 3, energy supply 104 may comprise a harvest circuit 124, comprising dipole antenna 126 connected to diode 128, with LED 130 connected thereacross. Such a harvest circuit 124 may be used independently of a triggering circuit, for example as described above with reference to and as illustrated in FIG. 2, and/or independently thereof.

It will be appreciated that triggering circuit 108 and harvest circuit 124 described above with reference to and shown in FIGS. 2 and 3 each are disclosed as a non-limiting example, and any suitable circuit may be provided, mutatis mutandis. It will also be appreciated that triggering circuit 108 and/or harvest circuit 124 may be modified based on the energy form used to trigger it, the energy form produced thereby, etc., mutatis mutandis.

According to some examples, triggering apparatus 300 is further configured to direct the energy supply to vary the energy level (e.g., intensity) produced by energy supply 104. Accordingly, a single miniature device 100 may be used to selectively produce different energy doses, for example to facilitate different treatments at a target site, vary the intensity of the therapeutic effect, etc.

According to some examples, drive portion 102 and energy supply 104 are formed as a monolithic unit, i.e., configured to remain together in the patient.

According to other examples, drive portion 102 is configured to separate from energy supply 104, for example under direction of driving apparatus 200 and/or triggering apparatus 300.

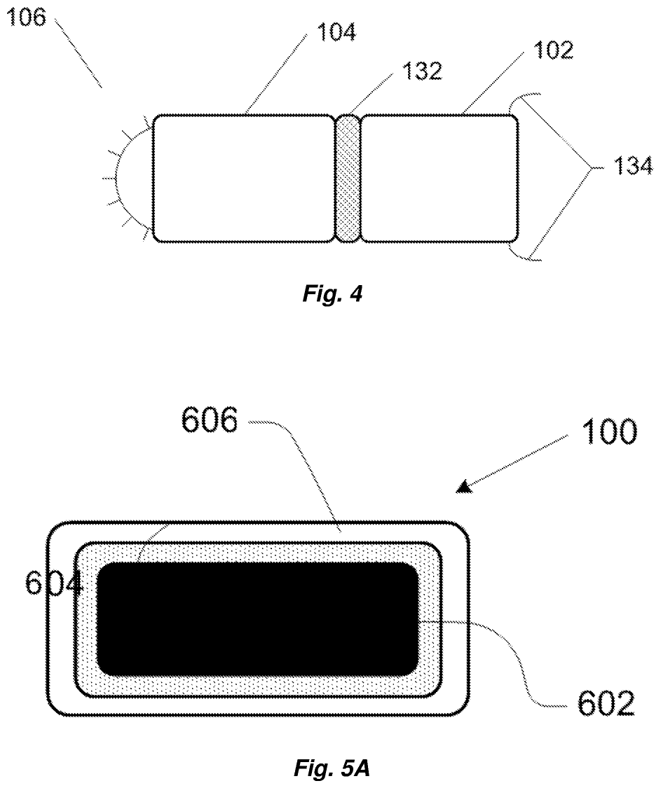

Drive portion 102 may be connected to energy supply 104 in any suitable manner. In some embodiments, for example as shown in FIG. 4, drive portion 102 is attached to energy supply 104 using adhesive material 132. Adhesive material 132 is configured to be disrupted under one or more predetermined conditions. The predetermined condition may be melting, dissolving in a solvent, chemically induced matrix rupture, exposure to radio, ultrasound waves, exposure to near infrared frequency, or a combination thereof. Adhesive material 132 may be insulated from the environment by a bioerodible material, thereby delaying the disruption of the adhesive material.

In some examples, energy supply 104, by itself or with drive portion 102, is configured to be anchored adjacent to the target site. Accordingly, miniature device 100, e.g., energy supply 104, may comprise one or more anchors 134 configured to grip, e.g., selectively, tissue or other suitable matter within the patient. In examples where drive portion 102 is configured to separate from energy supply 104, miniature device 100 may be maneuvered into a suitable position, anchors 134 may grip the patient's tissue thereby anchoring the energy supply at a suitable position adjacent the target site, the drive portion and energy supply separate, allowing drive portion 102 to be maneuvered elsewhere (e.g., to be retrieved by the user), while the energy supply remains adjacent the target site. This may be used, e.g., to facilitate long-term treatment in which a catalyzing dose of energy is delivered, for example hours, days, weeks, months, etc., after energy supply 104 has been deployed as above.

It will be appreciated that while the term “adjacent” is a relative term, which may be dependent on several factors, in its present use it refers to a distance at which it may provide a targeted energy dose to a target site. Accordingly, the term “adjacent” may include near to as well as at the target site.

It will be appreciated that while driving apparatus 200 is described herein as creating a magnetic force to manipulate drive portion 102 of miniature device 100, this is by way of example only, and a system in which a different non-contact force is used for the manipulation may be provided, mutatis mutandis. Examples of such non-contact forces include, but are not limited to, magnetic, electromagnetic, ultrasound, radio-frequency, optical, and a combination thereof.

It will be further appreciated that while system 10 is described with reference to activation of a therapeutic agent, system 10 may be used without such an agent, for example by providing a dose of energy configured to trigger a physiological process in the patient (i.e., a physiological response), e.g., by the cells, tissue, etc., which facilitates the therapeutic effect. The process may be, but it not limited to, enhanced local pharmacokinetics, absorption, rupture of a physiological barrier (e.g., lipid bilayers, multilayered linings of organs, organ envelopes, blood-brain barrier, blood-tumor barrier), distribution, permeability, proliferation, differentiation, adhesion, motility, or combinations thereof.

According to some examples, enhancement of intracellular and/or intranuclear bioavailability of a gene therapy may be achieved by exposure to the energy dose. The gene therapy may comprise, but is not limited to one comprising oligonucleotide sequences (e.g., ASO, RNAi, siRNA, miRNA, shRNA, CRISPR-Cas9 components or analogs, viral delivery-based agents, and/or oncolytic viruses).

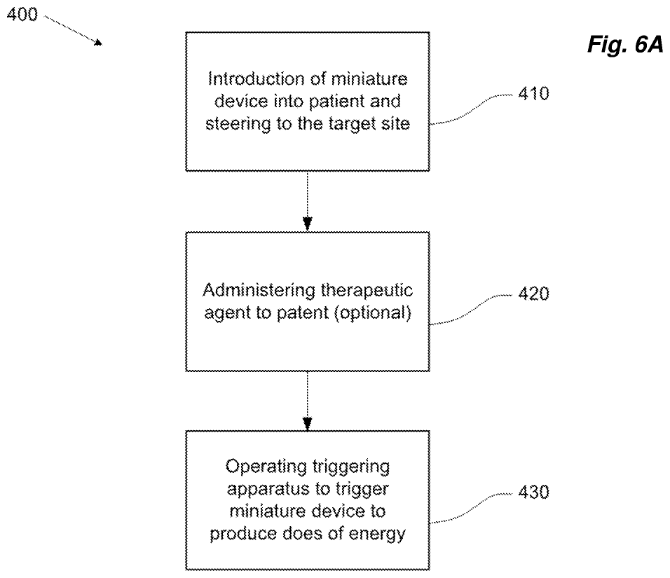

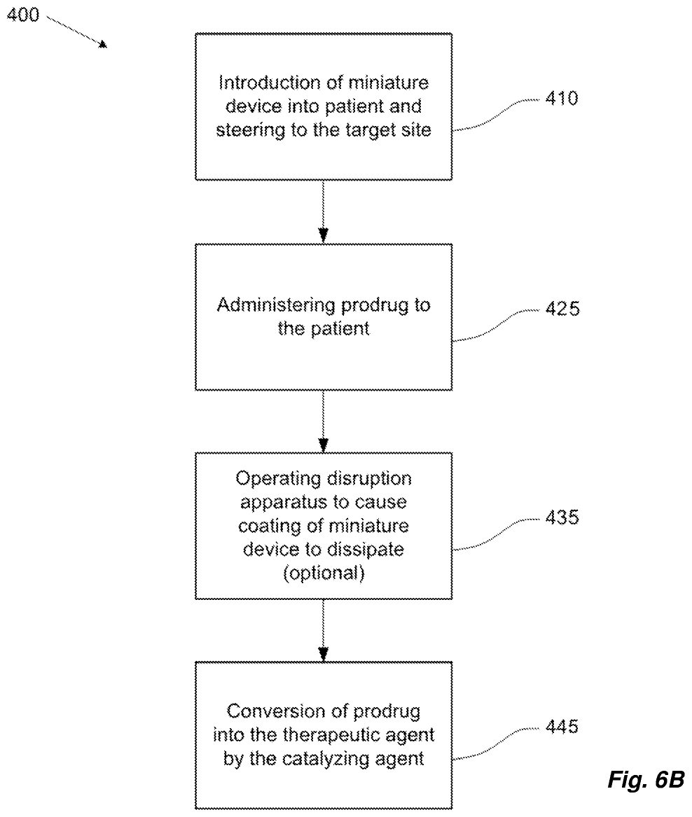

Accordingly, for example as illustrated in FIGS. 6A-6B, method 400 may be provided for using system 10, for example as described above with reference to and as illustrated in FIGS. 1A-1B. 2, 3, 4, 5A-5C, 12, and 13 for localized treatment of a patient at a target site in a patient.

In the block diagram provided in FIG. 6A, step 410 of method 400, miniature device 100 is introduced into the patient at an injection site being remote from the target site. The injection site may be, e.g., in the lumbar region of the spine, the cisterna magna adjacent the cerebellum, or at another suitable location. A user then operates driving apparatus 200, for example as is known in the art, to steer miniature device 100 to the target site in the patient. The target site may be, e.g., the midbrain, the basal ganglia, or any other suitable location.

In some examples, miniature device 100 may be maintained that the target site using driving apparatus 200. According to other examples, anchors 134 may be deployed to maintain energy supply 104 at a suitable location. When drive portion 102 and energy supply 104 are separatable from one another, drive portion 102 may be maneuvered out of the patient using driving apparatus 200.

In step 420 of method 400 shown in FIG. 6A, a therapeutic agent is administered to a patient. The therapeutic agent may be administered by any suitable method, for example locally or systemically, e.g., orally, intravenously, intramuscularly, subcutaneously, intranasally, sub-buccally, intrathecally, intracerebroventrally, etc. The therapeutic agent is configured to be activated by exposure to a predetermined energy dose, for example as described above, and remains inactive (e.g., dormant, inert, non-toxic, not bioavailable, etc.) until exposure to the predetermined energy dose.

It will be appreciated that step 420 is optional, for example when an energy dose is configured to trigger a process in the patient to facilitate a therapeutic effect step 420 may be omitted.

In step 430 of method 400, triggering apparatus 300 is operated to remotely trigger energy supply 104 of miniature device 100 to produce a suitable energy dose. According to examples in which a therapeutic agent is introduced into the patient, only the portion of the therapeutic agent in the vicinity of the energy supply is activated, thereby providing localized treatment. The remainder of the therapeutic agent, i.e., that which is not activated, is eliminated naturally by the patient.

For examples where an energy dose triggers a process in a patient to facilitate a therapeutic effect, only tissue in the vicinity of the energy supply is activated, thus providing localized treatment.

It will be appreciated that the steps of method 400 do not have to be fully carried out in the order presented, nor do they have to be carried out within a short time of one another. For example, depending on the amount of time which the therapeutic agent requires to reach the target site, step 420 (administering of agent) may be performed in advance of step 410 (delivery of miniature device 100 to the target site). According to other examples, miniature device 100, and in particular energy supply 104, may be delivered to the target site well in advance of treatment, with introduction of the therapeutic agent and/or production of the energy dose performed at one or more points in the future.

For another example as illustrated in FIG. 4B, a method 400 may be provided for using system 10, for example as described above with reference to and as illustrated in FIGS. 1B, 5A-5C, for localized treatment of a patient at a target site in a patient.

In step 410 of method 400, miniature device 100 is introduced into the patient at an injection site being remote from the target site. The injection site may be, e.g., in the lumbar region of the spine, the cisterna magna adjacent the cerebellum, or at another suitable location. A user then operates driving apparatus 200, for example as is known in the art, to steer miniature device 100 to the target site. The target site may be, e.g., the midbrain, the basal ganglia, or any other suitable location.

In step 425 of method 400, a prodrug is administered to the patient. The prodrug may be administered by any suitable method, for example locally or systemically, e.g., orally, intravenously, intramuscularly, subcutaneously, intranasally, sub-buccally, intrathecally, intracerebroventrally, etc. The therapeutic agent is converted into a therapeutic agent at the target site by the activating agent, directly or indirectly, and remains inactive or partially inactive (e.g., dormant, inert, non-toxic, not bioavailable) until exposure to the activating agent and/or auxiliary activating agent produced thereby.

In optional step 435 of method 400, disruption apparatus 300 is operated to create a condition at the target site which causes coating 606 to dissipate, thereby releasing the activating agent. Operation of triggering apparatus 300 may be such so that the activating agent is released at a predetermined rate, thereby controlling the level of the therapeutic effect, etc.

In step 445 of the method, the activating agent causes, directly or indirectly, conversion of the prodrug into the therapeutic agent, for as described above. (While step 445 may typically occur without outside intervention once suitable conditions have been established, it is presented herein as a step of the method for completeness.) Owing to the delivery of the activating agent only at the target site, only the portion of the prodrug which is in the vicinity of the activating agent is converted into the therapeutic agent, thereby providing localized treatment. The remainder of the prodrug, i.e., that which is not activated, is eliminated naturally by the patient.

It will be appreciated that the steps of method 400 do not have to be fully carried out in the order presented, nor do they have to be carried out within a short time of one another. For example, depending on the amount of time a therapeutic agent needs to reach a target site, step 425 (administering of a prodrug) is performed in advance of step 410 (delivery of miniature device 100 to a target site). According to other examples, miniature device 100 is delivered to a target site well in advance of treatment, with introduction of the prodrug performed at one or more points in the future.

It will be further appreciated that while herein the specification and appended claims reference is made to a therapeutic agent, in practice system 10 may be configured to facilitate treatment by more than one type of therapeutic agent, e.g., at the same time; the terms “therapeutic agent,” “activating agent.” “prodrug,” etc., are employed herein in the singular for simplicity of disclosure only, and are not to be construed as limiting any of the examples and/or embodiments disclosed or recited herein to a single therapeutic agent, etc., mutatis mutandis.

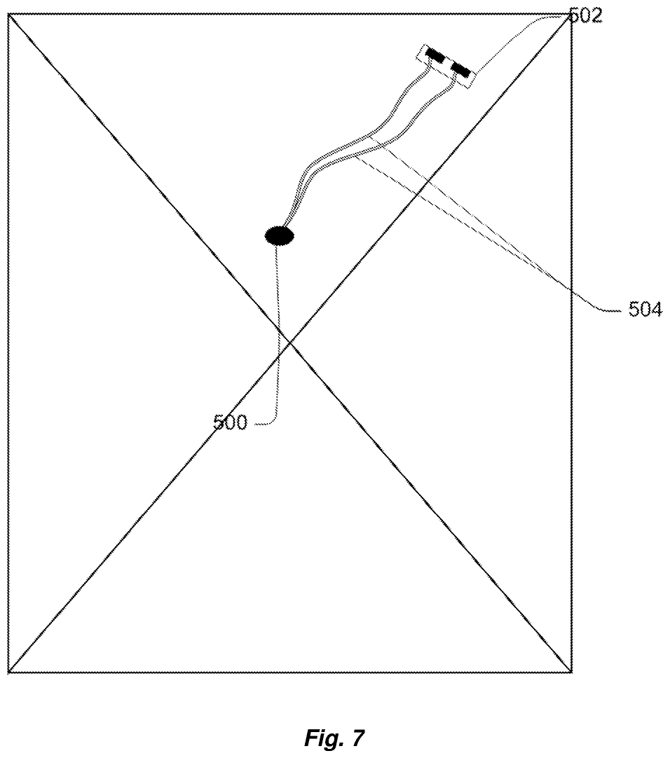

As illustrated in FIG. 7, there is provided a modification of system 10 described above with reference to FIGS. 1A-1B, 2, 3, 4, 5A-5C in which energy supply 500 is surgically implanted at or adjacent the target site. The energy supply 500 may be anchored in place using any suitable means.

According to some examples, a triggering apparatus, similar to that described above in connection with FIGS. 1A-1B, 2, 3, 4, 5A-5C, is provided to remotely trigger energy supply 500 to produce the catalyzing energy dose. According to some modifications, an interface 502 is implanted on the patient at a location accessible to a user, for example at the skin, and connected by one or more wires 504 to energy supply 500, to provide power and/or facilitate triggering of energy supply 500.

A method for localized treatment at a target site in a patient using the modified system described above with reference to and illustrated in FIG. 7 may be similar to as described above with reference to and as illustrated in FIGS. 4A-4B, mutatis mutandis.

It will be recognized that examples, embodiments, modifications, options, etc., described herein are to be construed as inclusive and non-limiting, i.e., two or more examples, etc., described separately herein are not to be construed as mutually exclusive of one another or in any other way limiting, unless such is explicitly stated and/or is otherwise clear. Those skilled in the art to which this invention pertains will readily appreciate that numerous changes, variations, and modifications can be made without departing from the scope of the presently disclosed subject matter, mutatis mutandis.

### EXAMPLES

A demonstration of the system of the present disclosure was performed in mouse, wherein the miniature delivery device delivered a payload recombinant adeno-associated virus (AAV) construct vector to a locus in either right hemisphere or left hemisphere brain.

Animals were grouped into negative control (no vector) and treated. In a first experiment, a firefly luciferase vector AAV1-CAG-LUCR was placed on miniature delivery devices (i.e., payload 604 of the present disclosure). The devices were suspended in solution at a density of about 1×10⁹ vp/μL and then injected into the mouse. A user steered the device to the animals' right hemispheres.

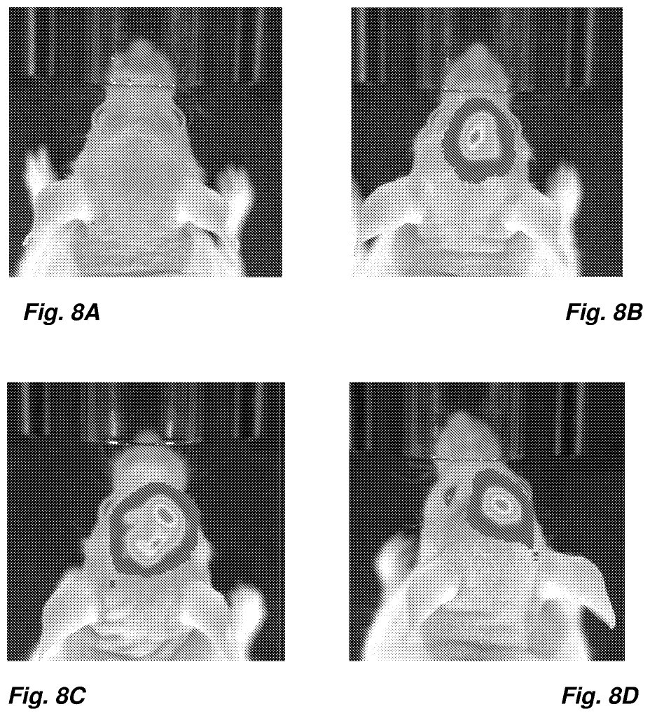

Mice were dosed daily with 1 μL or less of AAV-CAG-Luc (f), and expression levels were mapped by bioluminescence at day 7, day 11, day 20, and day 60 posttreatment. FIG. 8A depicts a negative control mouse having no bioluminescence. FIGS. 8B-8D depict bioluminescence local to right hemisphere at day 7 posttreatment, indicating expression of firefly luciferase.

In another experiment, mice were injected with two separate constructs having two different luciferase homologs to demonstrate localization efficacy. Aimed for the brain right hemisphere were miniature devices such as in the system of FIG. 1B loaded with a payload 606 of AAV1-LUC (Renilla) and aimed for the brain left hemisphere were miniature devices loaded with payload 606 of AAV1-LUCR (Firefly). The devices were suspended in solution at a density of about 1×10⁹ vp/μL and then injected into the mouse. A user steered the device loaded with AAV1-LUC (Renilla) to the right hemisphere, and then steered the device loaded with AAV1-LUCR (Firefly) to the left hemisphere.

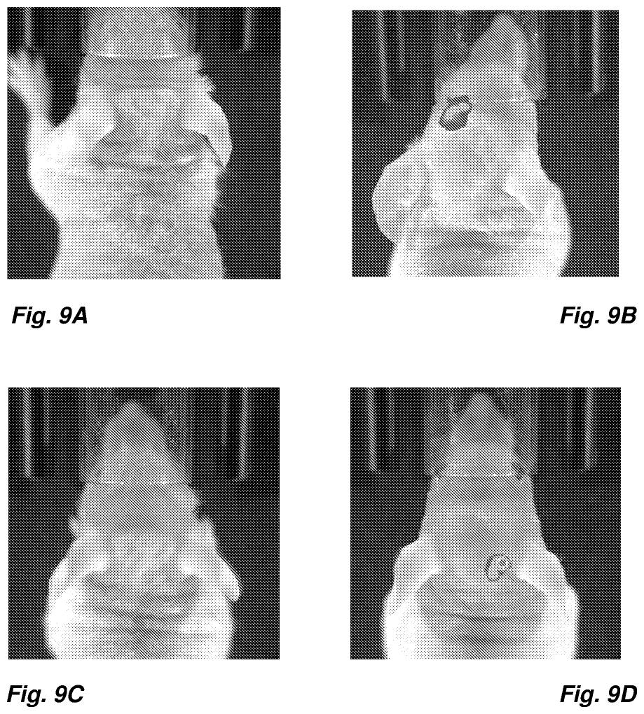

The mice were then dosed daily with 1 μL or less of AAV-CAG-Luc (f), and expression levels were mapped by bioluminescence at day 7, day 11, day 20, and day 60 posttreatment. FIGS. 9A and 9C show the same negative control animal, imaged for Renilla luciferase (FIG. 9A) and firefly luciferase (FIG. 9C) at day 11 posttreatment. FIG. 9B shows imaging for Renilla luciferin luminescence, which can be seen localized around the left side of the head; FIG. 9D shows imaging for firefly luciferin luminescence, which can be seen localized around the right side of the head.

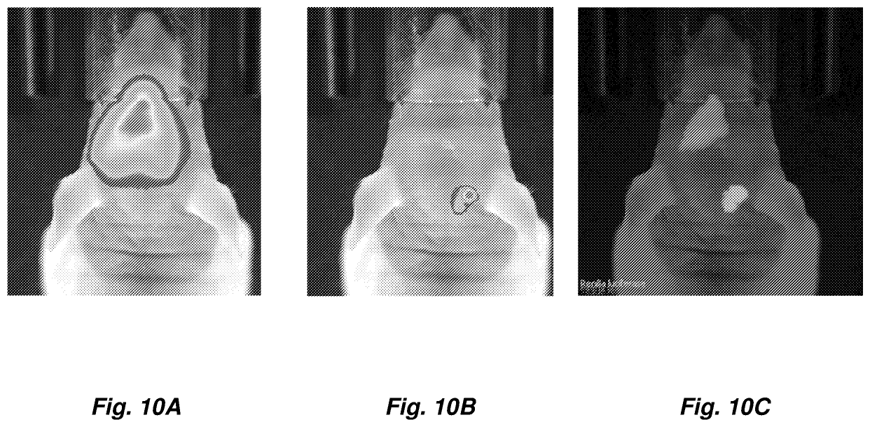

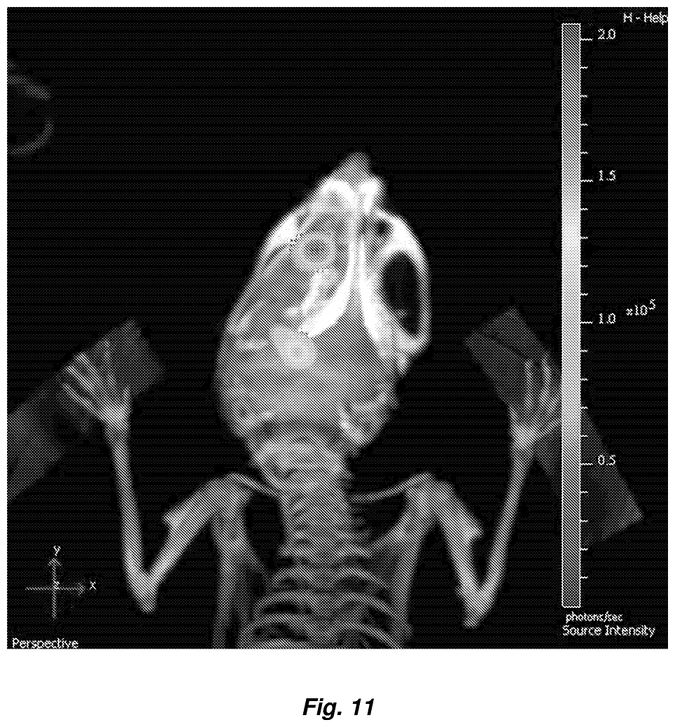

FIGS. 10A-10C depict the same individual specimen as in FIGS. 9B, 9D, imaged in day 60 within 1 hour of Renilla luciferin and firefly luciferin injection. FIG. 10C is an overlay composite showing respective localizations. FIG. 11 is an x-ray showing the 3-dimensional spatial separation of Renilla luminescence and firefly luminescence.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments. These and other changes can be made to the embodiments in light of the above-detailed description.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A system configured to facilitate treatment at a target site in a patient, the system comprising:
at least one miniature device configured to be maneuvered to the target site under manipulation by an external non-contact force, the miniature device comprising an externally triggered energy supply;
a driving apparatus configured for creating the external non-contact force to manipulate the miniature device to move within the patient; and
a triggering apparatus configured for remotely triggering the energy supply to produce a catalyzing dose of energy to induce a therapeutic effect at the target site.

2. The system according to claim 1, being configured to facilitate said treatment by a therapeutic agent, wherein the therapeutic agent is configured to produce the therapeutic effect in the presence of the catalyzing dose of energy.

3. The system according to claim 2, said therapeutic agent being configured to be photoactivated.

4. The system according to claim 3, wherein the therapeutic agent comprises a photosensitizing agent; a photocleavable moiety, and/or a molecule that assumes an active conformation, assembly, aggregation, and/or modification upon exposure to light.

5. The system according to claim 1, wherein said catalyzing dose of energy triggers a physiological process in the patient that facilitates the therapeutic effect.

6. The system according to claim 5, wherein the process is selected from the group consisting of enhanced local pharmacokinetics, absorption, rupture of a physiological barrier, distribution, permeability, proliferation, differentiation, adhesion, motility, and a combination thereof.

7. The system according to claim 1, said miniature device further comprising a drive portion affixed to the energy supply and configured to interact with the external non-contact force to effect maneuvering, and said drive portion being configured to separate from the energy supply.

8. The system according to claim 7, wherein the triggering and/or driving apparatus is configured to direct the separation of the drive portion and energy supply of the miniature device.

9. The system according to claim 7, wherein the drive portion is affixed to the energy supply by an adhesive material that is configured to be disrupted under a predetermined condition, thereby separating the carrier portion from the drive portion.

10. The system according to claim 9, wherein the predetermined condition is selected from the group consisting of melting, dissolving in a solvent, chemically induced matrix rupture, exposure to radio and/or ultrasound waves, and exposure to near infrared frequency.

11. The system according to claim 9, further comprising the therapeutic agent, and wherein disruption of the adhesive material releases the therapeutic agent.

12. The system according to claim 11, wherein said adhesive material is mixed with the therapeutic agent.

13. The system according to claim 9, wherein the adhesive material is insulated from the environment by a bioerodible material configured to delay the disruption of the adhesive material.

14. The system according to claim 1, wherein said miniature device comprises one or more anchors configured to anchor the energy supply adjacent the target site.

15. The system according to claim 1 wherein the miniature device carries a prodrug activating agent to facilitate the conversion of a prodrug into a therapeutic agent.

16. The system according to claim 15, wherein the system is further configured to produce the therapeutic effect in the presence of a catalyzing dose of energy.

17. The system according to claim 15, wherein the prodrug activating agent comprises a viral vector.

18. The system according to claim 15, wherein the miniature device further comprises a coating configured to at least partially dissipate at the target site under one or more predetermined conditions, thereby releasing the prodrug activating agent.

19. A system configured to facilitate treatment by a therapeutic agent at a target site in a patient, said therapeutic agent being formed by conversion of a prodrug, the system comprising:
at least one miniature device configured to be maneuvered to the target site under manipulation by an external non-contact force, the miniature device carrying an activating agent that converts the prodrug into the therapeutic agent;
a driving apparatus configured for creating the external non-contact force to manipulate the miniature device to move within the patient.

20. The system according to claim 19, wherein said activating agent converts the prodrug into the therapeutic agent by directly interacting with the prodrug.

21. The system according to claim 19, wherein the activating agent comprises an enzyme and/or an enzymatically active oligonucleotide.

22. The system according to claim 19, wherein said activating agent encodes an auxiliary activating agent, and said auxiliary activating agent converts the prodrug into the therapeutic agent by directly interacting with the prodrug.

23. The system according to claim 22, wherein an extracellular, intracellular, and/or intranuclear process expresses the auxiliary activating agent encoded by the activating agent.

24. The system according to claim 22, wherein the activating agent comprises an enzyme precursor, an oligonucleotide precursor, and/or a protease precursor.

25. The system according to claim 22, further comprising a vector configured for cellular delivery of the activating agent.

26. The system according to claim 25, wherein the vector is selected from a group including an adeno-associated virus, a human immunodeficiency virus, a human papillomavirus sequence, one or more small molecules, one or more lipids, a peptide sequence, and a recognition sequence.

27. The system according to claim 19, wherein the activating agent is and/or encodes one or more enzymes selected from a kinase, a phosphatase, a peptidase, a ligase, a lyase, a hydrolase, a protease, a deacetylase, a phosphodiesterase, an esterase, an amidase, a reductase, a phospholipase, or a cytochrome.

28. The system according to claim 19, wherein said activating agent is carried on an exterior surface of the miniature device.

29. The system according to claim 19, wherein said miniature device further comprises a coating configured to at least partially dissipate at the target site under one or more predetermined conditions, thereby releasing the activating agent.

30. The system according to claim 29, wherein the coating surrounds the activating agent.

31. The system according to claim 29, wherein the activating agent is mixed with the material of the coating.

32. The system according to claim 29, wherein at least one of the predetermined conditions is selected from dissolving, dispersion, decomposition, metabolism, a change in pH, a redox reaction, or the presence of one or more enzymes.

33. The system according to claim 19, wherein the miniature device is configured to controllably release the activating agent.

34. A method for providing localized treatment at a target site in a patient, the method comprising:
providing a system according to claim 1;
introducing the miniature device of the system into the patient at an injection site;
operating the driving apparatus of the system to navigate the miniature device to the target site; and
operating the triggering apparatus to trigger the miniature device to produce a catalyzing dose of energy to induces a therapeutic effect at the target site.

35. The method according to claim 34, further comprising administering a therapeutic agent to the patient, wherein said therapeutic agent produces the therapeutic effect in the presence of the catalyzing dose of energy.

36. A method for providing localized treatment at a target site in a patient, the method comprising:
providing a system according to claim 19;
introducing the miniature device of the system into the patient at an injection site;
operating the driving apparatus of the system to navigate the miniature device to the target site; and
administering a prodrug to the patient, wherein said prodrug is converted by the activating agent into the therapeutic agent to effect the treatment at the target site.
